(19) United States
(12) Patent Application Publication
(10) Pub. No.: US 2003/0089035 A1
(43) Pub. Date: May 15, 2003

(54) Method and composition for stimulating plant root growth

(76) Inventor: Jacques Courtemanche, Montreal (CA)

Correspondence Address: Jacques COURTEMANCHE, c/o PROTECTIONS EQUINOX INT'L INC., Suite 224, 4480, Cote-de-Liesse, Montreal, QC, H4N 2R1, CA

(21) Appl. No.: 09/986,825

(22) Filed: Nov. 13, 2001

(51) Int. Cl. A01B 79/02; A01H 3/00; A01C 1/00; A01G 1/00

(52) U.S. Cl. 47/58.1 SC

(57) ABSTRACT

Disclosed herein is a method and composition for stimulating the growth of plant roots. This composition includes manure produced by soil-consuming worms after having exclusively consumed an aerated soil mixture and is 100% natural.

### FIELD OF THE INVENTION

The present invention relates to the growth plant of plant roots. More specifically, the present invention is concerned with a method, a composition and a method of making such a composition for stimulating the growth of plant roots.

### BACKGROUND OF THE INVENTION

Root plant growth stimulators are well known. The majority of products actually used on the market in root stimulating processes are synthetic hormones, in powder or gel. These synthetic products are toxic and strong enough that they should not be used with nutritional plants.

There thus remains a need to provide a plant growth stimulator that is efficient yet non-toxic.

### OBJECTS OF THE INVENTION

It is therefore a general object of the present invention to provide an improved method and composition for stimulating the growth of plant roots.

An advantage of the present composition for stimulating plant root growth is that it is more efficient than existing synthetic products.

Another advantage of the present composition for stimulating plant root growth is that it is non-toxic and 100% natural.

Other objects and advantages of the present invention will become apparent from a careful reading of the detailed description provided herein, within appropriate reference to the accompanying drawings.

### SUMMARY OF THE INVENTION

More specifically, in accordance with an aspect of the present invention, there is provided a method of making a plant root growth stimulator, said method comprises the steps of:

a) feeding an aerated soil mixture to soil-consuming worms so as to produce manure; and

b) separating said manure from said worms, said manure forming said root growth stimulator.

Preferably, the method further comprises the steps of:

c) dehydrating said manure;

d) grinding said manure so as to obtain a fine powder, said powder forming said root growth stimulator;

e) adhering said powder to a cutting of a plant; and

f) planting the cutting in an agriculturally acceptable germination mixture.

Preferably, the step e) includes moisturizing the cutting of the plant and dipping the moisturized plant stem in said powder.

Preferably, the soil-consuming worms are Eudrelus eugenia type of worm.

Preferably, the aerated soil mixture is substantially free of any impurities.

Alternatively, the aerated soil mixture includes seaweed.

Preferably, the step a) includes mixing said worms in said soil mixture for a period equal to the time necessary for said worms to have totally consumed said soil mixture.

Alternatively, the manure is a first amount of manure, said method further comprises the steps of:

c) re-feeding said worms with an aerated soil mixture including seaweed so as to produce a second amount of manure;

d) separating said second amount of manure from said worms;

e) mixing said second amount of manure with said first amount of manure;

f) dehydrating said first and second amounts of manure; and

g) grinding said first and second amount of manure so as to produce a fine powder, said powder forming said root growth stimulator.

Preferably, the germination mixture includes worm manure, and further includes the ingredients selected from the group consisting of peat moss, seaweed, sand and combinations thereof.

Preferably, the aerated soil mixture is substantially free of chemical products.

In accordance with another aspect of the present invention, there is provided a composition for stimulating the root growth of plants, said composition comprises manure produced by soil-consuming worms after having exclusively consumed an aerated soil mixture.

Preferably, the aerated soil mixture is substantially free of chemical products, whereby said composition being 100% natural, and the manure is in a dry fine powder form.

### DETAILED DESCRIPTION OF THE PREFERRED EMBODIMENTS

A preferred embodiment in accordance with the present invention will be described herein.

The present invention provides a method of making a plant root growth stimulator.

The method according to the invention includes first feeding an aerated soil mixture to soil-consuming worms. Preferably, the aerated soil mixture is substantially free of impurities soil such as chemical product including without limitation insecticide, pesticide, and the like. The type of soil-consuming worms used in accordance with the present invention is Eudrelus eugenia.

Preferably, the African worm Eudrelus eugenia, which feeds almost exclusively on muck soil or the like may be bred so as to be used within the context of the present invention.

First, the soil-consuming worms are fed with aerated soil so as to produce manure. In one particular example of the present invention a quantity of approximately one (1) cup of worms/eight (8) liters pail of prepared and aerated soil is mixed from five (5) to ten (10) days and preferably for seven (7) days. At the end of this period, the worms have substantially consumed all of the soil such that only manure remains in the pail.

The manure will then be separated from the worms. In one embodiment the residue in the pail is processed in a “crypt” to separate the worms from the manure.

Alternatively, the worms may be re-fed with the same soil mixture and preferably seaweed so as to produce a second quantity of manure.

The manure will then be dehydrated. This may be accomplished by using a dehydrator of the type used to dehydrate fine herbs and the like. Of course, all other methods of dehydrating the manure may be contemplated within the scope of the invention.

The dehydrated manure will then be preferably ground down so as to obtain a fine powder that is easy to handle and use. For example, once the dehydration process is completed, the manure may be ground using a grinder of the type used for wheat to obtain a very fine powder.

This powder will be used to stimulate the growth of plant roots.

The powder is adhered to a stem, cutting or leave of the plant. For example the stem of the plant may be moistened and then dipped in the fine ground powder which will adhere to the stem.

The root is then planted in an agriculturally acceptable germination mixture. This germination mixture may include the worm manure as described above. Other ingredients may be selected from peat moss, seaweed and a little sand or any combination thereof.

Depending of the plant type, the roots will develop more or less rapidly. In the case of basil and cucumber plants for example, they will develop roots within approximately four (4) days. At that time, the plant “and its roots” can be planted in a more appropriate mixture.

When the soil mixture used to feed the worms is free form any non-natural chemical product, the produced composition is 100% natural and therefore “bio” certified.

The produced composition of the present invention can also be used as an efficient, and natural if applicable, fertilizer and the like for any plant.

### EXAMPLE 1

It is known that an apple seed almost never germinates in its natural state before drying for en extended period of time. However, when using the method disclosed herein, that is by moistening the seed and dipping it in the powder of the present invention before sowing it in a germination mixture, two (2) of the five (5) sown seeds were shown to develop roots and new apple trees were thus grown.

Preferably, the manure used in the composition for stimulating the root growth of plants according to the present invention comprises between 25% and 45% of dry matter, non-nutritive such as litter like matter or the like, between 40% and 95% of dry organic matter with an estimated Carbon/Nitrogen (C/N) ratio varying between five (5) and twenty (20), depending of the muck soil used, its humidity level and possible presence of seaweed mixed therewith.

Although the present method and composition for stimulating plant root growth have been described with a certain degree of particularity, it is to be understood that the disclosure has been made by way of example only and that the present invention is not limited to the features of the embodiments described and illustrated herein, but includes all variations and modifications within the scope and spirit of the invention as hereinafter claimed.

I claim:

1. A method of making a plant root growth stimulator, said method comprising the steps of:
a) feeding an aerated soil mixture to soil-consuming worms so as to produce manure; and
b) separating said manure from said worms, said manure forming said root growth stimulator.

2. A method according to claim 1, wherein said method further comprising the steps of:
c) dehydrating said manure; and
d) grinding said manure so as to obtain a fine powder, said powder forming said root growth stimulator.

3. A method according to claim 1, wherein said soil-consuming worms are Eudrelus eugenia type of worm.

4. A method according to claim 1, wherein said aerated soil mixture is substantially free of any impurities.

5. A method according to claim 1, wherein said aerated soil mixture includes seaweed.

6. A method according to claim 1, wherein step a) includes mixing said worms in said soil mixture for a period equal to the time necessary for said worms to have totally consumed said soil mixture.

7. A method according to claim 6, wherein said period is from five (5) to ten (10) days.

8. A method according to claim 7, wherein said period is equal to seven (7) days.

9. A method according to claim 1, wherein said manure is a first amount of manure, said method further comprising the steps of:
c) re-feeding said worms with an aerated soil mixture including seaweed so as to produce a second amount of manure;
d) separating said second amount of manure from said worms; and
e) mixing said second amount of manure with said first amount of manure to form said root growth stimulator.

10. A method according to claim 9, wherein said method further comprising the steps of:
f) dehydrating said first and second amounts of manure; and
g) grinding said first and second amount of manure so as to produce a fine powder, said powder forming said root growth stimulator.

11. A method of stimulating the root growth of a plant, said method comprising the steps of:
a) feeding an aerated soil mixture to soil-consuming worms so as to produce manure;
b) separating said manure from said worms;
c) dehydrating said manure;
d) grinding said manure so as to obtain a fine powder, said powder forming said root growth stimulator;
e) adhering said powder to a cuffing of a plant; and
f) planting the cutting in an agriculturally acceptable germination mixture.

12. A method according to claim 11, wherein step e) includes moisturizing the cutting of the plant and dipping the moisturized plant stem in said powder.

13. A method according to claim 11, wherein said germination mixture includes worm manure.

14. A method according to claim 13, wherein said germination mixture further includes the ingredients selected from the group consisting of peat moss, seaweed, sand and combinations thereof.

15. A method according to claim 11, wherein said soil-consuming worms are Eudrelus eugenia type of worm.

16. A method according to claim 11, wherein said aerated soil mixture is substantially free of chemical products.

17. A method according to claim 11, wherein said aerated soil mixture includes seaweed.

18. A composition for stimulating the root growth of plants, said composition comprising manure produced by soil-consuming worms after having exclusively consumed an aerated soil mixture.

19. A composition according to claim 18, wherein said manure is in a dry fine powder form.

20. A composition according to claim 18, wherein said aerated soil mixture is substantially free of chemical products, whereby said composition being 100% natural.
